SEQUENCE LISTING (8 sequences)

SEQ ID NO: 1 - protein, 7 aa, Artificial Sequence
  Description of Artificial Sequence Synthetic peptide
GFDFTTY

SEQ ID NO: 2 - protein, 6 aa, Artificial Sequence
  Description of Artificial Sequence Synthetic peptide
HPDSST

SEQ ID NO: 3 - protein, 10 aa, Artificial Sequence
  Description of Artificial Sequence Synthetic peptide
LYFGFPWFAY

SEQ ID NO: 4 - protein, 11 aa, Artificial Sequence
  Description of Artificial Sequence Synthetic peptide
KASQDVGTSVA

SEQ ID NO: 5 - protein, 7 aa, Artificial Sequence
  Description of Artificial Sequence Synthetic peptide
WTSTRHT

SEQ ID NO: 6 - protein, 8 aa, Artificial Sequence
  Description of Artificial Sequence Synthetic peptide
QQYSLYRS

SEQ ID NO: 7 - protein, 654 aa, Artificial Sequence
  Description of Artificial Sequence Synthetic polypeptide
EVQLVESGGGVVQPGRSLRLSCSASGFDFTTYWMSWVRQAPGKGLEWIGEIHPDSSTINYAPSLKDRFTISRDNAKNTLFLQMDSLRPEDTGVYFCASLYFGFPWFAYWGQGTPVTVSSGGGGSGGGGSGGGGSDIQLTQSPSSLSASVGDRVTITCKASQDVGTSVAWYQQKPGKAPKLLIYWTSTRHTGVPSRFSGSGSGTDFTFTISSLQPEDIATYYCQQYSLYRSFGQGTKVEIKRESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKMFWVLVVVGGVLACYSLLVTVAFIIFWVRSKRSRGGHSDYMNMTPRRPGPTRKHYQPYAPPRDFAAYRSGGGRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 8 - protein, 702 aa, Homo sapiens
MESPSAPPHRWCIPWQRLLLTASLLTFWNPPTTAKLTIESTPFNVAEGKEVLLLVHNLPQHLFGYSWYKGERVDGNRQIIGYVIGTQQATPGPAYSGREIIYPNASLLIQNIIQNDTGFYTLHVIKSDLVNEEATGQFRVYPELPKPSISSNNSKPVEDKDAVAFTCEPETQDATYLWWVNNQSLPVSPRLQLSNGNRTLTLFNVTRNDTASYKCETQNPVSARRSDSVILNVLYGPDAPTISPLNTSYRSGENLNLSCHAASNPPAQYSWFVNGTFQQSTQELFIPNITVNNSGSYTCQAHNSDTGLNRTTVTTITVYAEPPKPFITSNNSNPVEDEDAVALTCEPEIQNTTYLWWVNNQSLPVSPRLQLSNDNRTLTLLSVTRNDVGPYECGIQNELSVDHSDPVILNVLYGPDDPTISPSYTYYRPGVNLSLSCHAASNPPAQYSWLIDGNIQQHTQELFISNITEKNSGLYTCQANNSASGHSRTTVKTITVSAELPKPSISSNNSKPVEDKDAVAFTCEPEAQNTTYLWWVNGQSLPVSPRLQLSNGNRTLTLFNVTRNDARAYVCGIQNSVSANRSDPVTLDVLYGPDTPIISPPDSSYLSGANLNLSCHSASNPSPQYSWRINGIPQQHTQVLFIAKITPNNNGTYACFVSNLATGRNNSIVKSITVSASGTSPGLSAGATVGIMIGVLVGVALI

(19) United States
(12) Patent Application Publication
(10) Pub. No.: US 2021/0236548 A1
(43) Pub. Date: Aug. 5, 2021

(54) TREATMENT OF PROSTATE CANCER USING CHIMERIC ANTIGEN RECEPTORS

(71) Applicant: THE REGENTS OF THE UNIVERSITY OF CALIFORNIA, Oakland, CA (US)

(72) Inventors: Owen N. WITTE, Sherman Oaks, CA (US); John K. LEE, Santa Clarita, CA (US); Nathanael J. BANGAYAN, Los Angeles, CA (US)

(21) Appl. No.: 17/049,008

(22) PCT Filed: Apr. 22, 2019
(86) PCT No.: PCT/US2019/028577

§ 371 Date: Oct. 19, 2020

Related U.S. Application Data
(60) Provisional application No. 62/660,864, filed on Apr. 20, 2018.

(51) Int. Cl. A61K 35/17; C07K 14/705; A61P 35/00

(52) U.S. Cl. CPC A61K 35/17; A61P 35/00; C07K 14/70521

(57) ABSTRACT

Provided herein are methods of treating neuroendocrine prostate cancer (NEPC) with immune cells comprising a CEA-CAM5 chimeric antigen receptor (CAR). Also provided are methods of reducing or eliminating NEPC cancer cells with immune cells comprising a CEACAM5 CAR. Also provided are methods of treating a cancer with a molecular signature that is similar to a molecular signature of NEPC (e.g., small cell lung cancer (SCLC), small cell carcinoma of the pancreas (SCCP), or small cell prostate cancer).

### CROSS REFERENCE TO RELATED APPLICATIONS

This application claims priority to U.S. Provisional Patent Application No. 62/660,864, filed Apr. 20, 2018, the entire contents of which are incorporated by reference herein.

### STATEMENT REGARDING FEDERALLY SPONSORED RESEARCH OR DEVELOPMENT

This invention was made with government support. The government has certain rights in the invention.

### BACKGROUND

Prostate cancer is the most common non-skin cancer diagnosed in men and the second leading cause of cancer death in men (See Siegel R L et al., CA Cancer J Clin., 2016, 66:7-30). Over 95% of prostate cancers are diagnosed as prostate adenocarcinoma (PrAd), which is often characterized by glandular epithelial architecture, expression of luminal cytokeratins (CK8 and CK18), and active androgen receptor (AR) signaling. In advanced disease, blockade of AR signaling has been the mainstay of treatment for decades but inevitably leads to resistance in the form of castration-resistant prostate cancer (CRPC). Recent data indicate that CRPC can retain the PrAd histology or recur as a distinct subtype called neuroendocrine prostate cancer (NEPC). Recent work also indicates that a subset of CRPC assumes a double-negative (AR-negative, neuroendocrine-negative) phenotype that is maintained by enhanced FGF and MAPK pathway signaling (See Bluemn E G, et al., Cancer Cell, 2017, 32:474-489). NEPC comprises a group of neuroendocrine tumors that includes aggressive variants such as large cell carcinoma and small cell carcinoma of the prostate (See Epstein J I, et al., Am J Surg Pathol., 2014, 38:756-767). Aggressive NEPC evolves from PrAd following treatment in up to 20% of CRPC cases through neuroendocrine transdifferentiation which involves epigenetic reprogramming mediated by Polycomb proteins (See Clermont P L, et al., Clin Epigenetics, 2016, 8:16 and Kleb B, et al., Epigenetics, 2016, 11:184-193) and often the loss of the tumor suppressors RB1 and TP53 (See Ku S Y, et al., Science, 2017, 355:78-83). NEPC often exhibits an anaplastic morphology, expression of neuroendocrine markers including chromogranins and synaptophysin, loss of AR signaling, overexpression and amplification of MYCN and AURKA (See Beltran H, et al., Cancer Discov, 2011, 1:487-495; Lee J K, et al., Cancer Cell, 2016, 29:536-547; and Dardenne E, et al., Cancer Cell, 2016, 30:563-577), resulting in a particularly poor prognosis due to rapid and progressive metastatic dissemination.

CEACAM5 (carcinoembryonic antigen-related cell adhesion molecule 5) is a glycophosphatidylinositol-anchored membrane protein and established tumor antigen whose expression has primarily been associated with adenocarcinomas of the colon, rectum, and pancreas. Despite case reports of detectable serum CEACAM5 in rare patients with advanced prostate cancer, a systematic study of CEACAM5 IHC in prostate tumors identified no expression in both primary and metastatic samples (See Blumenthal R D et al., BMC Cancer, 2007, 7:2).

### SUMMARY

The present disclosure provides methods of treating a subject having neuroendocrine prostate cancer (NEPC), comprising administering to the subject an infusion of immune cells comprising a chimeric antigen receptor (CAR) comprising a CEACAM5 antigen-binding moiety, a transmembrane domain, and an immune cell activation moiety, wherein the immune cell activation moiety comprises one or more signaling domains. In some embodiments, the present disclosure provides a method of treating neuroendocrine prostate cancer, wherein the neuroendocrine prostate cancer is CEACAM5⁺ neuroendocrine prostate cancer.

In certain embodiments, provided herein are methods of treating a subject having CEACAM5⁺ neuroendocrine prostate cancer, comprising administering an infusion of immune cells, wherein the immune cells are CD8⁺ T cells, and the immune cells comprise a CAR comprising a CEACAM5 scFv antigen-binding moiety, a spacer domain having a length of 200 to 300 amino acids, a transmembrane domain, and an immune cell activation moiety comprising one or more signaling domains.

In some embodiments, the present disclosure provides a method of reducing or eliminating NEPC cancer cells, comprising contacting the NEPC cancer cells with an infusion of immune cells comprising a chimeric antigen receptor (CAR) comprising a CEACAM5 antigen-binding moiety, a transmembrane domain, and an immune cell activation moiety, wherein the immune cell activation moiety comprises one or more signaling domains. In certain embodiments, the NEPC cancer cells comprise CEACAM5⁺ NEPC cancer cells.

In certain embodiments, the present disclosure provides a method of treating a subject with small cell cancer, comprising administering an infusion of immune cells comprising a chimeric antigen receptor (CAR) comprising a CEACAM5 antigen-binding moiety, a transmembrane domain, and an immune cell activation moiety, wherein the immune cell activation moiety comprises one or more signaling domains. In certain embodiments, a small cell cancer can include at least one of lung, prostate, pancreas, and stomach small cell cancer. In certain embodiments, the small cell cancer is CEACAM5 positive.

In some embodiments, the present disclosure provides a method of reducing or eliminating small cell cancer cells, comprising contacting the small cell cancer cells with an infusion of immune cells comprising a chimeric antigen receptor (CAR) comprising a CEACAM5 antigen-binding moiety, a transmembrane domain, and an immune cell activation moiety, wherein the immune cell activation moiety comprises one or more signaling domains. In certain embodiments, a small cell cancer can include at least one of lung, prostate, pancreas, and stomach small cell cancer. In certain embodiments, the small cell cancer is CEACAM5 positive.

In certain embodiments, the method comprises administering an infusion of immune cells including T cells. In certain embodiments, the method comprises administering an infusion of T cells including CD3⁺ T cells. In certain embodiments, the method comprises administering an infusion of T cells including CD8⁺ T cells.

In certain embodiments, the method comprises administering an infusion of immune cells including natural killer (NK) cells. In certain embodiments, the method administering an infusion of immune cells including natural killer T (NKT) cells.

In certain embodiments of the present disclosure, immune cells administered for treating a subject with cancer (e.g., NEPC, small cell lung cancer (SCLC), small cell carcinoma of the pancreas (SCCP), or small cell prostate cancer) comprises a chimeric antigen receptor (CAR). In certain embodiments, a CAR comprises a CEACAM5 antigen-binding moiety. In some embodiments, CEACAM5 antigen-binding moiety comprises an antibody or antigen-binding fragment thereof. In certain embodiments, the antibody or antigen-binding fragment of CEACAM5 antigen-binding moiety comprises the CDRs of labetuzumab. In certain embodiments, the antibody or antigen-binding fragment of CEACAM5 antigen-binding moiety comprises: a VH-CDR1 comprising the sequence set forth in SEQ ID NO:1; a VH-CDR2 comprising the sequence set forth in SEQ ID NO:2; a VH-CDR3 comprising the sequence set forth in SEQ ID NO:3; a VL-CDR1 comprising the sequence set forth in SEQ ID NO:4; a VL-CDR2 comprising the sequence set forth in SEQ ID NO:5; and a VL-CDR3 comprising the sequence set forth in SEQ ID NO:6. In certain embodiments, the antigen-binding fragment is a Fab or an scFv. In certain embodiments, the antigen-binding fragment is an scFv. In certain embodiments, the antigen-binding fragment is an scFv derived from labetuzumab.

In certain embodiments of the present disclosure, immune cells administered for treating a subject with cancer (e.g., NEPC, small cell lung cancer (SCLC), small cell carcinoma of the pancreas (SCCP), or small cell prostate cancer) comprises a chimeric antigen receptor (CAR). In certain embodiments, a CAR comprises a transmembrane domain. In certain embodiments, the transmembrane domain is a CD28 transmembrane domain or a CD8a transmembrane domain. In certain embodiments, the transmembrane domain is a CD28 transmembrane domain.

In certain embodiments of the present disclosure, immune cells administered for treating a subject with cancer (e.g., NEPC, small cell lung cancer (SCLC), small cell carcinoma of the pancreas (SCCP), or small cell prostate cancer) comprises a chimeric antigen receptor (CAR). In certain embodiments, a CAR comprises an immune cell activation moiety. In certain embodiments, the immune cell activation moiety comprises one or more signaling domains. In certain embodiments, the immune cell activation moiety comprises one or more co-stimulatory domains and an immunoreceptor tyrosine-based activation motif (ITAM)-containing signaling domain. In certain embodiments, co-stimulatory domains include a CD28 co-stimulatory domain, a 4-1BB co-stimulatory domain, an OX40 co-stimulatory domain, or an ICOS co-stimulatory domain. In certain embodiments, the immune cell activation moiety comprises a CD28 co-stimulatory domain. In certain embodiments, the immune cell activation moiety comprises a 4-1BB co-stimulatory domain. In certain embodiments, the immune cell activation moiety comprises CD28 and 4-1BB co-stimulatory domains. In certain embodiments, the immune cell activation moiety comprises an ITAM-containing signaling domain. In certain embodiments, the ITAM-containing signaling domain comprises a CD3ζ signaling domain or an FcRγ signaling domain. In certain embodiments, the ITAM-containing signaling domain comprises a CD3ζ signaling domain. In certain embodiments, the immune cell activation moiety comprises a 28-ΔIL2RB-z(YXXQ) domain.

In certain embodiments of the present disclosure, immune cells administered for treating a subject with cancer (e.g., NEPC, small cell lung cancer, small cell carcinoma of the pancreas, or small cell prostate cancer) comprises a chimeric antigen receptor (CAR). In certain embodiments, a CAR comprises a spacer domain. In some embodiments, the spacer domain has a length of 1 to 500 amino acids. In some embodiments, the spacer domain has a length of 200 to 300 amino acids. In some embodiments, the spacer domain has a length of 229 amino acids. In certain embodiments, the spacer domain comprises a hinge domain from an immunoglobulin. In certain embodiments, the hinge domain from an immunoglobulin comprises the hinge domain from IgG1, IgG2, IgG3, or IgG4. In certain embodiments, the hinge domain from an immunoglobulin comprises the hinge domain from human IgG4. In certain embodiments, the spacer domain comprises the CH2-CH3 domain from an immunoglobulin. In certain embodiments, the spacer domain comprises a hinge domain from an immunoglobulin and the CH2-CH3 domain from an immunoglobulin. In certain embodiments, the spacer domain comprises the extracellular domain of CD8a. In certain embodiments, the spacer domain comprises a hinge domain from an immunoglobulin and the extracellular domain of CD8a.

In certain embodiments, provided herein are methods of treating a subject with CEACAM5⁺ neuroendocrine prostate cancer comprising administering an infusion of immune cells (e.g., CD8⁺ T cells) comprising a CAR comprising a CEACAM5 scFv antigen-binding moiety, a spacer domain having a length of 200 to 300 amino acids, a transmembrane domain, and an immune cell activation moiety comprising one or more signaling domains.

In certain embodiments of the present disclosure, immune cells administered for treating a subject with cancer (e.g., NEPC, small cell lung cancer (SCLC), small cell carcinoma of the pancreas (SCCP), or small cell prostate cancer) comprises a chimeric antigen receptor (CAR). In certain embodiments, a CAR comprises an scFv derived from labetuzumab, a hinge of human IgG4, a CH2-CH3 domain of an immunoglobulin, a CD28 transmembrane domain, a CD28 co-stimulatory domain, and a CD3ζ signaling domain. In certain embodiments, the CH2-CH3 domain is a human IgG4 CH2-CH3 domain. In certain other embodiments, the CAR comprises the amino acid sequence set forth in SEQ ID NO:7. In certain embodiments, the CAR comprises an scFv derived from labetuzumab, a hinge of human IgG4, a CH2-CH3 domain of an immunoglobulin, a CD28 transmembrane domain, a 4-1BB co-stimulatory domain, and a CD3ζ signaling domain. In certain other embodiments, the CAR comprises the amino acid sequence set forth in SEQ ID NO:7. In certain embodiments, the CAR comprises an scFv derived from labetuzumab, a hinge of human IgG4, a CH2-CH3 domain of an immunoglobulin, a CD28 transmembrane domain, a CD28 co-stimulatory domain, a 4-1BB co-stimulatory domain, and a CD3ζ signaling domain. In certain embodiments, the CAR stimulates interferon gamma (IFNγ) release by the immune cells. In certain embodiments, the immune cells are autologous immune cells. In certain embodiments, the immune cells are allogeneic immune cells. In certain embodiments, the immune cells are administered intravenously.

### BRIEF DESCRIPTION OF THE DRAWINGS

These and other features, aspects, and advantages of the present invention will become better understood with regard to the following description, and accompanying drawings.

FIG. 1 shows the immunoblot analysis of select PrAd (LNCaP, CWR22Rv1, and DU145) and NEPC (NCI-H660, MSKCC EF1, and LASCPC-01) cell lines as well as benign human tissues (brain, heart, kidney, liver, and lung) with antibodies against STEAP1, FXYD3, FOLH1, NCAM1, SNAP25, and CEACAM5. Antibody against GAPDH was used as a loading control.

FIG. 2 shows flow cytometry histogram plots of the PrAd cell line LNCaP and the NEPC cell line NCI-H660 stained with antibodies against STEAP1, FXYD3, NCAM1, and CEACAM5. The peak on the right-hand side indicates the positive population.

FIG. 3 represents the schematic of the chimeric antigen receptor (CAR) construct targeting CEACAM5 (scFv=single chain variable fragment, TM=CD28 transmembrane domain, CS=co-stimulatory domain).

FIGS. 4A and 4B show interferon-y (IFN-γ) quantitation in the media after co-culture of short spacer CEACAM5 CAR-transduced, long spacer CEACAM5 CAR-transduced, or untransduced T cells with CEACAM5-negative or CEACAM5-positive target cell lines as shown. FIG. 4A shows the interferon-y (IFN-γ) quantitation 12 hours after co-culture. FIG. 4B shows the interferon-y (IFN-γ) quantitation 24 hours after co-culture. Standard error measurements for 4 replicate wells are displayed. Data are representative of 3 independent experiments with similar results. ns represents non-significance and **** represents p<0.0001 by two-way ANOVA statistical analysis.

FIGS. 5A and 5B show relative viability over time of target cells co-cultured with long spacer CEACAM5 CAR-transduced T cells. FIG. 5A shows the relative viability of CEACAM5-negative MSKCC EF1 target cells co-cultured with long spacer CEACAM5 CAR-transduced T cells. FIG. 5B shows the relative viability of CEACAM5-positive NCI-H660 target cells co-cultured with long spacer CEACAM5 CAR-transduced T cells. Effector-to-target ratios varying from 1:5 to 2:1 are shown. Standard error measurements for 3 replicate wells at each time point are displayed. Data are representative of 2 independent experiments with similar results.

FIGS. 6A and 6B show specificity of the cytotoxic activity of CEACAM5 CAR T cells in an engineered CEACAM5-positive prostate cancer cell line. FIG. 6A presents interferon-γ (IFN-γ) quantitation in the media at 24 and 48 hours after co-culture of long spacer CEACAM5 CAR-transduced or untransduced T cells with CEACAM5-negative DU145 target cells or CEACAM5-positive DU145-CEACAM5 target cell lines at a 1:1 effector-to-target ratio. Standard error measurements for 3 replicate wells are displayed. ns represents non-significance and **** represents p<0.0001 by two-way ANOVA statistical analysis. FIG. 6B presents relative viability over time of CEACAM5-negative DU145 target cells and engineered CEACAM5-positive DU145-CEACAM5 target cells co-cultured with long spacer CEACAM5 CAR-transduced T cells at a 1:1 effector-to-target ratio. Standard error measurements for 3 replicate wells at each timepoint are displayed.

FIGS. 7A and 7B illustrate the schematic description of additional chimeric antigen receptor (CAR) constructs targeting CEACAM5 (scFv=single chain variable fragment, TM=CD28 transmembrane domain, CS=co-stimulatory domain).

FIG. 8 shows interferon-γ (IFN-γ) quantitation in the media after co-culture of various short spacer CEACAM5 CAR-transduced, long spacer CEACAM5 CAR-transduced, or untransduced T cells with CEACAM5-negative or CEACAM5-positive DU145 target cell lines. Long spacer-CS1=Anti-CEACAM5-long spacer-CD28-CD3ζ); short spacer-CS2=Anti-CEACAM5-short spacer-4-1BB-CD3ζ); long spacer-052=Anti-CEACAM5-long spacer-4-1BB-CD3ζ); short spacer-CS3=Anti-CEACAM5-short spacer-CD28-4-1BB-CD3ζ); long spacer-CS3=Anti-CEACAM5-long spacer-CD28-4-1BB-CD3ζ). Standard error measurements for 3 replicate wells at each timepoint are displayed.

FIG. 9 shows viability over time of engineered CEACAM5-positive DU145-CEACAM5 target cells co-cultured with different long spacer CEACAM5 CAR (CD28, 4-1BB, or CD28-4-1BB co-stimulatory domains)-transduced T cells at a 1:1 effector-to-target ratio. Standard error measurements for 3 replicate wells at each timepoint are displayed.

### DETAILED DESCRIPTION

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antigen” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. The present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated, synthesized or can be derived from a biological sample, or might be macromolecule besides a polypeptide. Such a biological sample can include, but is not limited to a tissue sample, a tumor sample, a cell or a fluid with other biological components.

The term “co-stimulatory molecule” refers to the cognate binding partner on a T cell that specifically binds with a co-stimulatory ligand, thereby mediating a co-stimulatory response by the T cell, such as, but not limited to, proliferation. Co-stimulatory molecules are cell surface molecules other than antigen receptors or their ligands that are required for an efficient immune response. Co-stimulatory molecules include, but are not limited to an MHC class 1 molecule, BTLA and a Toll ligand receptor, as well as OX40, CD2, CD27, CD28, CDS, ICAM-1, LFA-1 (CD11a/CD18) and 4-1BB (CD137).

A co-stimulatory domain can be the intracellular portion of a co-stimulatory molecule. A co-stimulatory molecule can be represented in the following protein families: TNF receptor proteins, Immunoglobulin-like proteins, cytokine receptors, integrins, signaling lymphocytic activation molecules (SLAM proteins), and activating NK cell receptors. Examples of such molecules include CD27, CD28, 4-1BB (CD137), OX40, GITR, CD30, CD40, ICOS, HVEM, lymphocyte function-associated antigen-1 (LFA-1), CD2, CD7, LIGHT, NKG2C, SLAMF7, NKp80, CD160, B7-H3, and a ligand that specifically binds with CD83, and the like.

The term “autologous” refers to any material derived from the same individual to whom it is later to be re-introduced into the individual.

CEACAM5

CEACAM5 is also known as Carcinoembryonic Antigen Related Cell Adhesion Molecule 5. The RefSeq accession number for CEACAM5 is NM_004363.5 as shown on the NCBI website as of Apr. 10, 2018. The amino acid sequence of human CEACAM5, transcript variant 1 is shown in the table below.

| Name | Amino Acid Sequence |
|---|---|
| CEACAM5 | MESPSAPPHR WCIPWQRLLL TASLLTFWNP PTTAKLTIES |
| (human) | TPFNVAEGKE VLLLVHNLPQ HLFGYSWYKG ERVDGNRQII |
| | GYVIGTQQAT PGPAYSGREI IYPNASLLIQ NIIQNDTGFY |
| | TLHVIKSDLV NEEATGQFRV YPELPKPSIS SNNSKPVEDK |
| | DAVAFTCEPE TQDATYLWWV NNQSLPVSPR LQLSNGNRTL |
| | TLFNVTRNDT ASYKCETQNP VSARRSDSVI LNVLYGPDAP |
| | TISPLNTSYR SGENLNLSCH AASNPPAQYS WFVNGTFQQS |
| | TQELFIPNIT VNNSGSYTCQ AHNSDTGLNR TTVTTITVYA |
| | EPPKPFITSN NSNPVEDEDA VALTCEPEIQ NTTYLWWVNN |
| | QSLPVSPRLQ LSNDNRTLTL LSVTRNDVGP YECGIQNELS |
| | VDHSDPVILN VLYGPDDPTI SPSYTYYRPG VNLSLSCHAA |
| | SNPPAQYSWL IDGNIQQHTQ ELFISNITEK NSGLYTCQAN |
| | NSASGHSRTT VKTITVSAEL PKPSISSNNS KPVEDKDAVA |
| | FTCEPEAQNT TYLWWVNGQS LPVSPRLQLS NGNRTLTLFN |
| | VTRNDARAYV CGIQNSVSAN RSDPVTLDVL YGPDTPIISP |
| | PDSSYLSGAN LNLSCHSASN PSPQYSWRIN GIPQQHTQVL |
| | FIAKITPNNN GTYACFVSNL ATGRNNSIVK SITVSASGTS |
| | PGLSAGATVG IMIGVLVGVA LI |

The CEACAM5 gene encodes a cell surface glycoprotein that is a member of the carcinoembryonic antigen (CEA) family of proteins. The encoded protein has been used as a clinical biomarker for certain gastrointestinal cancers and may promote tumor development through its role as a cell adhesion molecule. Additionally, the encoded CEACAM5 protein may regulate differentiation, apoptosis, and cell polarity. This relevant gene coding sequence is present in a CEA family gene cluster on chromosome 19. Alternative splicing results in multiple transcript variants of CEACAM5.

In some embodiments, provided herein are methods of treating a subject with a cancer that has elevated expression of CEACAM5 relative to a control. The control can be, e.g., normal tissue that is of the same developmental origin as the relevant tumor tissue. The control expression level of CEACAM5 can also be a pre-determined threshold level (See Lee et al, Proc Natl Acad Sci USA. 2018 May 8; 115(19)). Methods for assessing CEACAM5 expression are well-known in the art and can include flow cytometry, immunoassays, and/or RT-PCR. In some embodiments, provided herein are methods of treating a subject with a cancer that is CEACAM5+ (e.g., pancreatic cancer, small cell carcinoma of the pancreas (SCCP), lung cancer, small-cell lung cancer (SCLC), prostate cancer, small cell prostate cancer, small cell neuroendocrine carcinoma, stomach cancer, colorectal cancer, and cervical cancer).

Chimeric Antigen Receptors (CARs)

In some aspects, a chimeric antigen receptor (CAR) provided herein comprises a CEACAM5 antigen-binding moiety, a transmembrane domain, and an immune cell activation moiety, wherein the immune cell activation moiety comprises one or more signaling domains.

CEACAM5 Antigen Binding Moiety

In some embodiments, the CEACAM5 antigen-binding moiety comprises an antibody or antigen-binding fragment thereof.

In some embodiments, the antibody or antigen-binding fragment thereof comprises one or more or all of the CDRs of labetuzumab. In some embodiments, the antibody or antigen-binding fragment thereof comprises: a VH-CDR1 comprising the sequence set forth in SEQ ID NO:1; a VH-CDR2 comprising the sequence set forth in SEQ ID NO:2; a VH-CDR3 comprising the sequence set forth in SEQ ID NO:3; a VL-CDR1 comprising the sequence set forth in SEQ ID NO:4; a VL-CDR2 comprising the sequence set forth in SEQ ID NO:5; and a VL-CDR3 comprising the sequence set forth in SEQ ID NO:6. In some embodiments, the antibody or antigen-binding fragment thereof comprises one or more or all of the CDRs of an anti-CEACAM5 antibody described in International Pat. Pub. No. WO2014079886, which is incorporated by reference in its entirety for all purposes.

In some embodiments, the antigen-binding fragment is a Fab or an scFv. In some embodiments, the antigen-binding fragment is an scFv. In some embodiments, the antigen-binding fragment is an scFv derived from labetuzumab. In some embodiments, the scFv derived from labetuzumab is described in U.S. Pat. No. 5,874,540A, which is incorporated by reference in its entirety for all purposes. In one aspect such antibody fragments are functional in that they retain the equivalent binding affinity, e.g., they bind the same antigen with comparable affinity, as the IgG antibody from which they are derived. In one aspect such antibody fragments are functional in that they elicit a biological response that can include, but is not limited to, activation of an immune response, inhibition of signal-transduction resulting from binding of its target antigen, inhibition of kinase activity, and the like, as will be understood by a skilled artisan.

Transmembrane Domain

In some embodiments, a transmembrane domain anchors the CAR to the cell surface, and connects the extracellular ligand binding domain that confers target specificity (e.g., CEACAM5 antigen binding moiety) to the intracellular signaling domain (e g, immune cell activation moiety), thus impacting expression of the CAR on the cell surface. In certain embodiments, the transmembrane domain may be derived either from a natural or from a recombinant source. In certain embodiments, the domain may be derived from any membrane-bound or transmembrane protein. In one aspect, the transmembrane domain provides stability to the CAR molecule. A transmembrane domain of particular use in the present disclosure may include at least the transmembrane region(s) of e.g., the alpha, beta or zeta chain of the T-cell receptor, CD28, CD3 epsilon, CD45, CD4, CD5, CD8a, CD9, CD16, CD22, CD33, CD37, CD64, CD80, CD86, CD134, CD137, CD154.

In some embodiments, the transmembrane domain of a CAR is a CD28 transmembrane domain or a CD8a transmembrane domain. In some embodiments, the transmembrane domain is a CD28 transmembrane domain.

Spacer Domain

In some embodiments, the CAR further comprises a spacer domain between the antigen-binding moiety and the transmembrane domain.

In some embodiments, the spacer domain has a length of 1 to 500 amino acids, such as 1 to 50, 1 to 100, 100 to 200, 200 to 300, 300 to 400, or 400 to 500 amino acids. In some embodiments, the spacer domain has a length of 200 to 300 amino acids. In some embodiments, the spacer domain has a length of 200 to 250 amino acids. In some embodiments, the spacer domain has a length of 229 amino acids.

In some embodiments, the spacer domain comprises a hinge domain from an immunoglobulin. In some embodiments, the hinge domain from an immunoglobulin comprises the hinge domain from IgG1, IgG2, IgG3, or IgG4. In some embodiments, the hinge domain from an immunoglobulin comprises the hinge domain from human IgG1 or IgG4. In some embodiments, the hinge domain from an immunoglobulin comprises the hinge domain from human IgG4.

In some embodiments, the spacer domain comprises the CH2-CH3 domain from an immunoglobulin. In some embodiments, the spacer domain comprises a hinge domain from an immunoglobulin and the CH2-CH3 domain from an immunoglobulin. In some embodiments, the hinge domain from an immunoglobulin comprises the hinge domain from IgG1, IgG2, IgG3, or IgG4, and the CH2-CH3 domain from an immunoglobulin comprises the CH2-CH3 domain from IgG1, IgG2, IgG3, or IgG4.

In some embodiments, the spacer domain comprises the extracellular domain of CD8a. In some embodiments, the spacer domain comprises a hinge domain from an immunoglobulin and the extracellular of CD8a. In some embodiments, the hinge domain from an immunoglobulin comprises the hinge domain from IgG1, IgG2, IgG3, or IgG4.

Immune Cell Activation Moiety

In some embodiments, the immune cell activation moiety activates at least one of the normal effector functions of the immune cell. In some embodiments, effector function is a specialized function of a cell. In some embodiments, the immune cell activation moiety transduces the effector function signal and directs the cell to perform a specialized function. In certain embodiments, an immune cell activation moiety can also include the T cell receptor (TCR) and co-receptors that act in concert to initiate signal transduction following antigen receptor engagement.

In some embodiments, the immune cell activation moiety comprises one or more signaling domains. In some embodiments, the one or more signaling domains includes at least one of a co-stimulatory domain and of an immunoreceptor tyrosine-based activation motif (ITAM)-containing signaling domain. In some embodiments, the one or more signaling domains includes at least two of a co-stimulatory domain and one of an immunoreceptor tyrosine-based activation motif (ITAM)-containing signaling domain.

In some embodiments, the immune cell activation moiety comprises one or more co-stimulatory domains. In some embodiments, the co-stimulatory domain comprises a CD28 co-stimulatory domain, a 4-1BB co-stimulatory domain, an OX40 co-stimulatory domain, or an Inducible T-cell costimulator (ICOS) co-stimulatory domain. In some embodiments, the co-stimulatory domain comprises a CD28 co-stimulatory domain. In some embodiments, the co-stimulatory domain comprises a 4-1BB co-stimulatory domain. In some embodiments, the immune cell activation moiety comprises two co-stimulatory domains. In some embodiments, two co-stimulatory domains comprise a CD28 and a 4-1BB co-stimulatory domain. In some embodiments, two co-stimulatory domains comprise a CD28 and an OX40 co-stimulatory domain. In some embodiments, two co-stimulatory domains comprise a CD28 and an ICOS co-stimulatory domain.

In some embodiments, the immune cell activation moiety comprises an ITAM-containing signaling domain. In some embodiments, the ITAM-containing signaling domain comprises a CD3ζ signaling domain or an FcRγ signaling domain. In some embodiments, the ITAM-containing signaling domain comprises a CD3ζ signaling domain

In some embodiments, the immune cell activation moiety comprises a 28-ΔIL2RB-z(YXXQ) domain, which comprises a truncated cytoplasmic domain from the interleukin (IL)-2 receptor β-chain (IL-2Rβ) and a STATS-binding tyrosine-X-X-glutamine (YXXQ) motif, together with a CD3ζ signaling domain and a CD28 co-stimulatory domain. See Kagoya Y, et al., Nat Med, 2018, 24:352-359, incorporated by reference in its entirety for all purposes.

CEACAM5 CAR

In some embodiments, the CAR comprises an scFv derived from labetuzumab, a hinge of human IgG4, a CH2-CH3 domain of an immunoglobulin (e.g., IgG4), a CD28 transmembrane domain, a CD28 co-stimulatory domain, and a CD3ζ signaling domain. In some embodiments, the CAR comprises an scFv derived from labetuzumab, a hinge of human IgG4, a CH2-CH3 domain of an immunoglobulin (e.g., IgG4), a CD28 transmembrane domain, a 4-1BB co-stimulatory domain, and a CD3ζ signaling domain. In some embodiments, the CAR comprises an scFv derived from labetuzumab, a hinge of human IgG4, a CH2-CH3 domain of an immunoglobulin (e.g., IgG4), a CD28 transmembrane domain, a CD28 co-stimulatory domain, a 4-1BB co-stimulatory domain, and a CD3ζ signaling domain.

In some embodiments, the CAR comprises the amino acid sequence set forth in SEQ ID NO:7. In some embodiments, SEQ ID NO: 7 comprises a CAR comprising an scFv derived from labetuzumab, a hinge of human IgG4, a CH2-CH3 domain of an immunoglobulin, a CD28 transmembrane domain, a CD28 co-stimulatory domain, and a CD3ζ signaling domain.

In some embodiments, the CEACAM5 CAR T cells administered to the subject for the methods of treatment (e.g., NEPC, SCCP, SCLC) stimulate interferon gamma (IFNγ) release.

Immune Cells

In another aspect, immune cells are engineered to express the chimeric antigen receptors described herein. In some embodiments, the immune cells are T cells. T cells can be obtained from a number of sources, including peripheral blood mononuclear cells, bone marrow, lymph node tissue, cord blood, thymus tissue, tissue from a site of infection, ascites, pleural effusion, spleen tissue, and tumors. In some embodiments, the T cells are CD3⁺ T cells. In some embodiments, the T cells are CD8⁺ T cells such as cytotoxic T lymphocytes (CTLs).

In some embodiments, the immune cells are natural killer (NK) cells or natural killer T (NKT) cells.

In some embodiments, the immune cells are autologous immune cells. In some embodiments, the immune cells are allogeneic immune cells.

Methods of Treatment

In some aspects, provided herein are methods of treating a subject with neuroendocrine prostate cancer (NEPC). In certain embodiments, provided herein are methods of treating a subject with CEACAM5-positive NEPC. In some embodiments, provided herein are methods of treating a subject with a cancer that shares a similar molecular signature with NEPC. In some embodiments, the NEPC molecular signature comprises certain oncogenic drivers of NEPC.

In some embodiments, oncogenic drivers of NEPC include TP53, AKT1, RB1, BCL2, and c-Myc.

### TP53 (Tumor Protein P53)

This gene encodes a tumor suppressor protein containing transcriptional activation, DNA binding, and oligomerization domains. The encoded protein responds to diverse cellular stresses to regulate expression of target genes, thereby inducing cell cycle arrest, apoptosis, senescence, DNA repair, or changes in metabolism. Mutations in this gene are associated with a variety of human cancers. Alternative splicing of this gene and the use of alternate promoters result in multiple transcript variants and isoforms. Examples of human TP53 sequences are available under the reference sequence NM_000546.

### AKT1 (AKT Serine/Threonine Kinase I)

AKT1, also referred to as protein kinase B, is a known oncogene. AKT activation relies on the PI3K pathway, and is recognized as a critical node in the pathway. The E17 hotspot is the most characterized of AKT1 mutations, and has been shown to result in activation of the protein. Mutations in AKT1 have also been shown to confer resistance to allosteric kinase inhibitors in vitro. Multiple alternatively spliced transcript variants have been found for this gene. Examples of human AKT1 sequences are available under the reference sequence NM_005163.

### RB1 (RB Transcriptional Corepressor I)

RB1 is a Protein Coding gene. Diseases associated with RB1 include retinoblastoma and small cell lung cancer. The protein encoded by this gene is a negative regulator of the cell cycle. Examples of human RB1 sequences are available under the reference sequence NM_000321.

### BCL2 (Apoptosis Regulator)

This gene encodes an integral outer mitochondrial membrane protein that blocks the apoptotic death of some cells such as lymphocytes. Alternative splicing of this gene results in multiple transcript variants. Examples of human BCL2 sequences are available under the reference sequence NM_000633.

c-myc

This gene is a proto-oncogene and encodes a nuclear phosphoprotein that plays a role in cell cycle progression, apoptosis and cellular transformation. The encoded protein forms a heterodimer with the related transcription factor MAX. This complex binds to the E box DNA consensus sequence and regulates the transcription of specific target genes. Amplification of this gene is frequently observed in numerous human cancers. Examples of human c-myc sequences are available under the reference sequence NM_002467.5.

Small cell cancers generally share a small-round-blue-cell morphology, markers of neuroendocrine differentiation (e.g., chromogranin A, neural cell adhesion molecule 1, and synaptophysin), high proliferative indices, and an aggressive clinical course marked by rapid dissemination. In some embodiments, the present disclosure provides methods for treating small cell cancers that share a similar molecular signature as NEPC. In certain embodiments, small cell cancers that share a similar molecular signature as NEPC are CEACAM5-positive. In some embodiments, the oncogenic drivers that drive NEPC are the same oncogenic drivers that drive small cell cancers. In some embodiments, oncogenic drivers of small cell cancers include TP53, AKT1, RB1, BCL2, and c-Myc. In certain embodiments, small cell cancer includes small cell lung cancer (SCLC), small cell prostate cancer, small cell carcinoma of the pancreas (SCCP), and small cell neuroendocrine carcinoma.

In some embodiments, the present disclosure provides methods for treating a disease associated with CEACAM5-positive expression. In some embodiments, the present disclosure provides methods for treating a cancer that is CEACAM5 positive (e.g., pancreatic cancer, small cell carcinoma of the pancreas (SCCP), lung cancer, prostate cancer, small cell prostate cancer, small cell lung cancer (SCLC), small cell neuroendocrine carcinoma, stomach cancer, colorectal cancer, and cervical cancer).

The methods comprise administering an infusion of immune cells comprising a chimeric antigen receptor (CAR) comprising a CEACAM5 antigen-binding moiety, a hinge from an immunoglobulin, a transmembrane domain, and an immune cell activation moiety, wherein the immune cell activation moiety comprises one or more signaling domains such as intracellular signaling domains (e.g., from 4-1BB or CD3c). In some embodiments, the immune cells (e.g., T cells) are administered intravenously.

In some aspects, also provided herein are methods of reducing or eliminating NEPC cancer cells in a subject in need thereof. The methods comprise contacting NEPC cancer cells with engineered immune cells comprising a chimeric antigen receptor (CAR) comprising a CEACAM5 antigen-binding moiety, a hinge from an immunoglobulin, a transmembrane domain, and an immune cell activation moiety, wherein the immune cell activation moiety comprises one or more signaling domains. In some embodiments, the NEPC cancer cells comprise CEACAM5⁺ NEPC cancer cells.

In some embodiments, the reduction or elimination of NEPC cancer cells in a subject in need thereof is due to an anti-tumor immune response elicited by the CAR-modified T cells. In certain embodiments, the anti-tumor immune response elicited by the CAR-modified T cells may be an active or a passive immune response, or alternatively may be due to a direct vs indirect immune response.

Combination Therapies

A CAR described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A CAR described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

### EXAMPLES

The following are examples of methods and compositions of the invention. It is understood that various other embodiments may be practiced, given the general description provided herein.

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B(1992).

Materials and Methods

Cell lines. LNCaP, CWR22Rv1, and DU145 (ATCC) were grown in RPMI with 10% FBS. NCI-H660 (ATCC) and LASCPC-01 (Lee J K, et al., Cancer Cell, 2016, 29:536-547) were grown in HITES media containing RPMI, 5% FBS, 10 nM hydrocortisone, 10 nM beta-estradiol (Sigma), insulin-transferrin-selenium, and Glutamax (Life Technologies). MSKCC EF1 was derived from the organoid line MSKCC-CaP4 (Gao D, et al., Cell, 2014, 159:176-187) and was grown in RPMI with 10% FBS.

Flow Cytometry. LNCaP was non-enzymatically dissociated with Versene EDTA solution (Thermo Fisher Scientific). NCI-H660 was collected from suspension culture and dissociated mechanically by pipetting. Cell lines were washed with PBS and incubated in flow cytometry staining buffer (PBS with 2% FBS and 0.09% sodium azide) with primary antibody or isotype control antibodies for 1 h. Cells were washed with PBS and incubated with mouse or rabbit IgG (H+L) fluorescein-conjugated secondary antibody (R&D Systems) for 1 h. Cells were washed with PBS, resuspended in flow cytometry staining buffer, and analyzed on a BD FACSCanto (BD Biosciences).

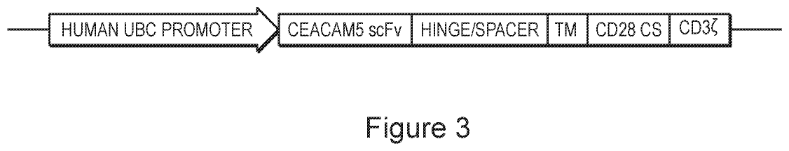

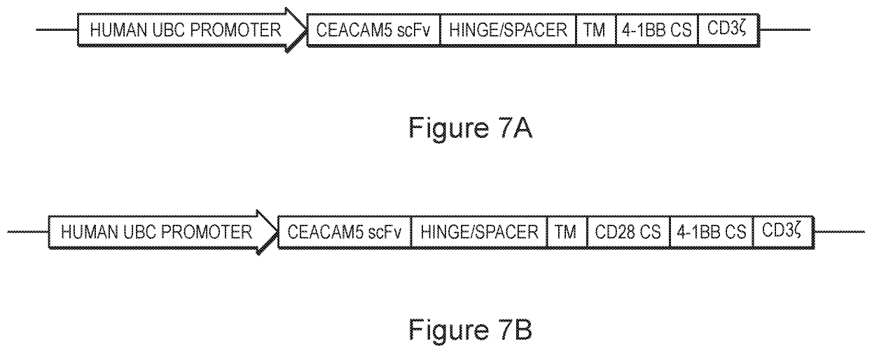

Lentiviral Vectors. The third-generation lentiviral vector FU-CGW, derived from FUGW, was used to label target cell lines with GFP for co-culture experiments. Human CEACAM5 cDNA was cloned into FU-CGW by NEBuilder HiFi DNA Assembly (New England Biolabs) to generate the lentiviral vector FU-CEACAM5-CGW to express CEACAM5 in select target cell lines. The short spacer and longer spacer CEACAM5 CAR constructs (described in FIG. 3, FIGS. 7A and 7B) were generated by NEBulder HiFi DNA Assembly of custom gBlocks gene fragments (Integrated DNA Technologies) and cloned into FU-W. Lentiviruses were produced and titered as previously described (Xin L, et al., Proc Natl Acad Sci USA, 2003, 100 Suppl 1:11896-11903, incorporated by reference in its entirety).

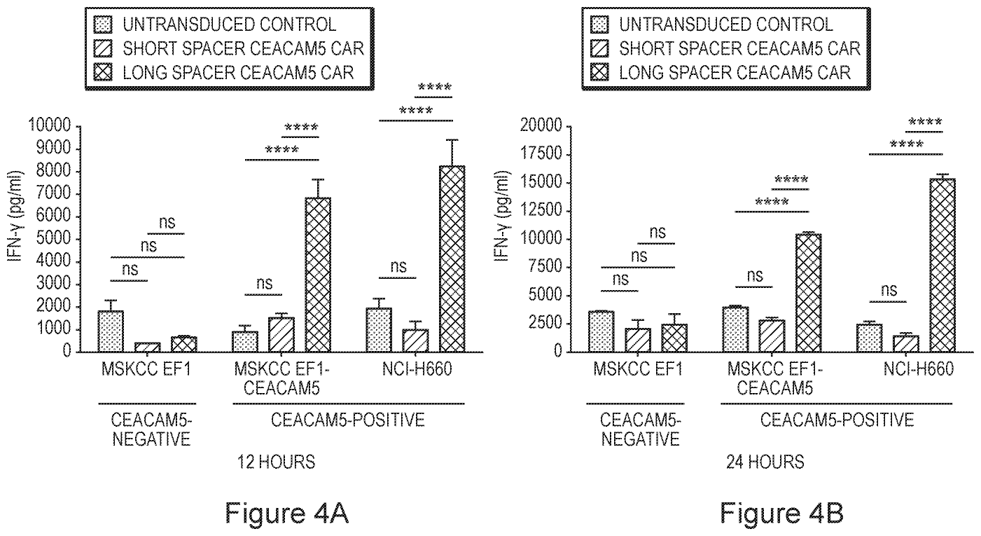

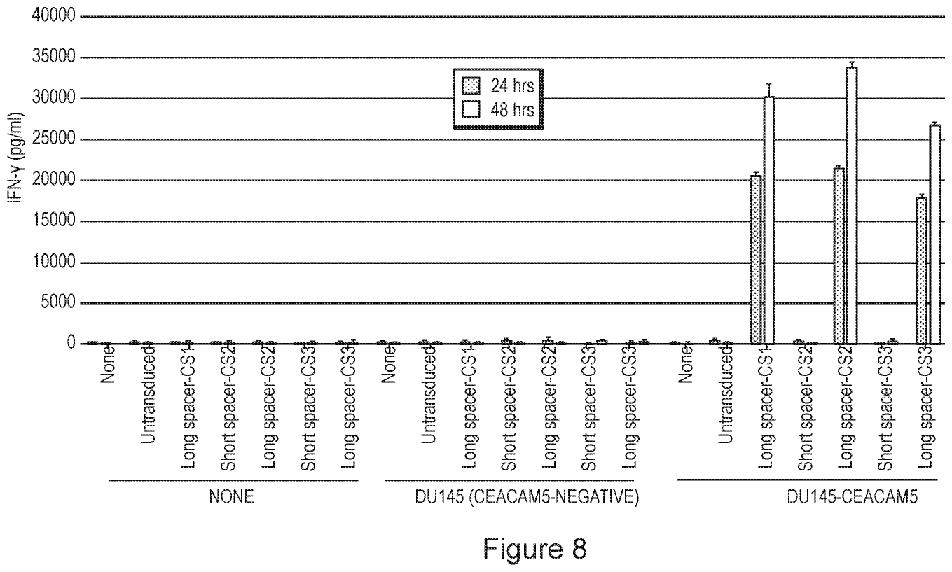

CAR T Cell Engineering and Co-culture Assays. Deidentified human PBMCs were obtained from the UCLA Virology Core Laboratory and grown in TCM base media composed of AIM V medium (Thermo Fisher Scientific), 5% heat-inactivated human AB serum, 2 mM glutamine, and 55 uM 2-mercaptoethanol (Sigma). For co-culture experiments involving interferon gamma release assays measured by ELISA, human PBMCs were activated in a 24-well plate coated with 1 ug/ml anti-CD3 (eBioscience OKT-3), 1 ug/ml anti-CD28 (eBioscience CD28.2), and 300 U/ml IL-2 in TCM base media. After 48 h, cells were spin infected daily for two days with various CEACAM5 CAR lentiviruses at an MOI of approximately 5-11 in TCM base media, 300 U/ml IL-2, and 8 ug/ml polybrene. After each infection, the cells were washed and grown in TCM base media with 300 U/ml IL-2. 96 h after final spin infection, T cell transduction efficiency was measure by flow cytometry and T cells were co-cultured with target cells at a target:effector ratio of 1:1. Supernatant was harvested at 12 and 24 h (to obtain experimental data shown in FIGS. 4A and 4B) or at 24 and 48 h (to obtain experimental data shown in FIG. 8) after co-culture. IFN-γ was quantitated with the BD OptEIA Human IFN-γ ELISA Set (BD Biosciences) according to the manufacturer's protocol. For co-culture experiments with direct visualization of cytotoxicity by live cell imaging, human PBMCs were activated with Gibco Dynabeads Human T-Activator CD3/CD28 (Thermo Fisher Scientific) in TCM base media with 50 U/ml IL-2 at a cell:bead ratio of 1. After 96 h, T cells were infected with CAR lentivirus by spin infection in TCM base media with 50 U/ml IL-2 at an MOI of 0.5-50. Cells were washed 24 h after infection and cultured in TCM base media with 50 U/ml IL-2. Dynabeads were removed 48 h after infection. 96 h after spin infection, T cell transduction efficiency was measured by flow cytometry and T cells were co-cultured with target cells at a range of target:effector ratios. Cytotoxicity was measured by Incucyte ZOOM through quantification of GFP-positive target cell counts.

### Example 1: Validation of CEACAM5 as a Target Antigen in NEPC

CEACAM5 was identified as a candidate NEPC target antigen by transcriptomic analysis of diverse prostate cancer datasets and by integrated transcriptomic and proteomic analysis of the prostate cancer cell lines (See Lee et al, Proc Natl Acad Sci USA. 2018 May 8; 115(19)).

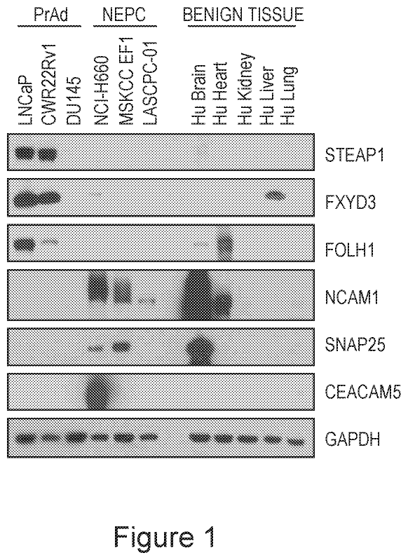

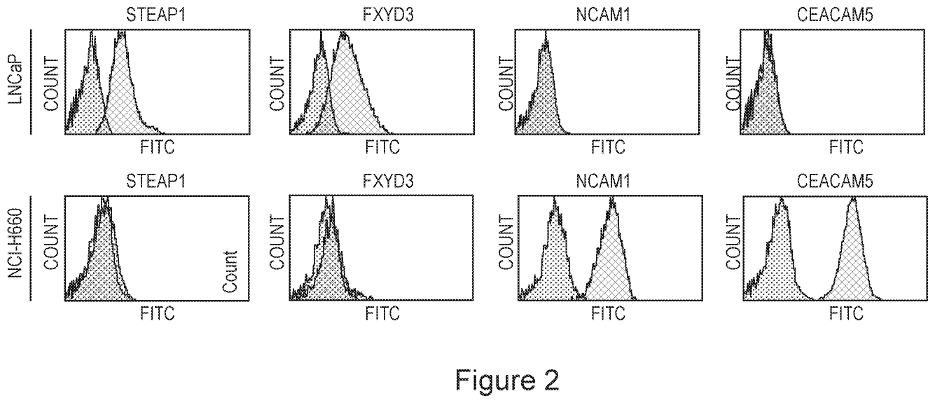

Of the candidates with high composite ranks, the PrAd-specific expression of STEAP1, FXYD3, and FOLH1 (PSMA) and the NEPC-specific expression of NCAM1, SNAP25, and CEACAM5 were validated by immunoblot (FIG. 1) and immunohistochemistry (IHC) of prostate cancer cell lines and xenografts (See Lee et al, Proc Natl Acad Sci USA. 2018 May 8; 115(19)). Flow cytometry confirmed the surface protein expression of STEAP1 and FXYD3 on the LNCaP PrAd line but not on the NCI-H660 NEPC line. Conversely, surface protein expression of NCAM1 and CEACAM5 were found on NCI-H660 but not on LNCaP. (FIG. 2)

The potential for CEACAM5-targeted therapy in NEPC was examined. The safety implications were determined by the systemic expression of CEACAM5 in normal human tissues at the mRNA and protein levels. Evaluation of the NIH GTEx database showed that CEACAM5 gene expression in men is limited to the colon, esophagus, and small intestine (See The Genotype-Tissue Expression (GTEx) project, Nature Genetics, 2013, 45:580-585, which is incorporated by reference in its entirety). In concordance with gene expression data from the GTEx database, immunoblot analysis of a range of human tissue lysates from vital organs revealed absence of CEACAM5 protein expression in the brain, heart, kidney, liver, and lung (FIG. 1). In addition, IHC of a normal human tissue microarray demonstrated CEACAM5 expression limited to the luminal lining of the colon and rectum in men.

These data indicate that CEACAM5 is a promising target antigen for therapeutic development in NEPC.

### Example 2: Therapeutic Targeting of CEACAM5 in NEPC

Two lentiviral CEACAM5 CAR constructs encoding a single chain variable fragment (scFv) derived from labetuzumab (See Stein R & Goldenberg D M, Mol Cancer Ther., 2004, 3:1559-1564, which is incorporated by reference in its entirety; other suitable anti-CEACAM5 antibodies are described in International Pat. Pub. No. WO2014079886, which is incorporated by reference in its entirety), hinge/spacer, CD28 transmembrane domain, CD28 co-stimulatory domain, and CD3 activation domain were generated(FIG. 3). The corresponding CDR sequences of labetuzumab are presented in SEQ ID NOs:1-6. The exemplary CEACAM5 CARs differed based on the presence of either a short spacer (IgG4 hinge) or a long spacer (IgG4 hinge and CH2+CH3 spacer). The CEACAM5 CAR with the long spacer has the amino acid sequence shown in SEQ ID NO:7. T cells expanded from human peripheral blood mononuclear cells were transduced with the CAR constructs and co-culture assays with target NEPC cell lines MSKCC EF1 (CEACAM5-negative, FIG. 1), MSKCC EF1-CEACAM5 (engineered to express CEACAM5), and NCI-H660 (CEACAM5-positive, FIG. 1 and FIG. 2) were performed at a fixed effector-to-target ratio of 1:1.

Analysis of the supernatant at 12 and 24 hours by interferon-gamma (IFN-γ) ELISA revealed enhanced antigen-specific IFN-γ release associated with the long spacer CEACAM5 CAR (FIGS. 4A and 4B). In contrast the short spacer CEACAM5 CAR did not increase the antigen-specific IFN-γ release, indicating that a longer spacer is useful for optimal target binding and T cell activation under the experimental conditions tested.

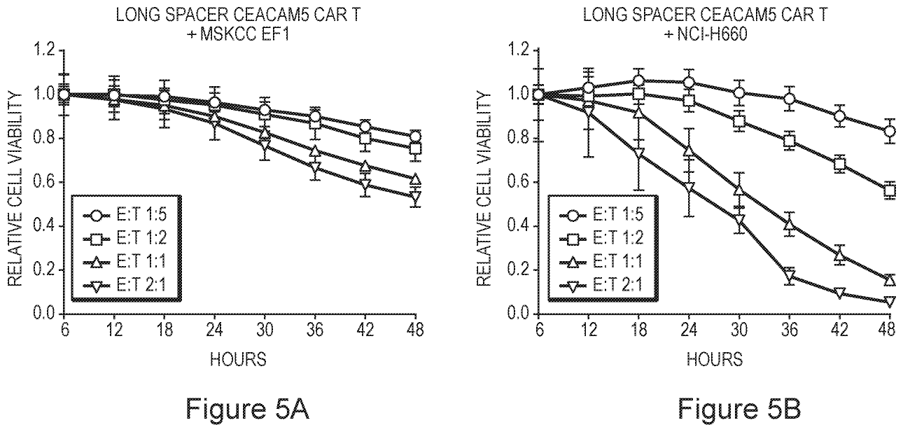

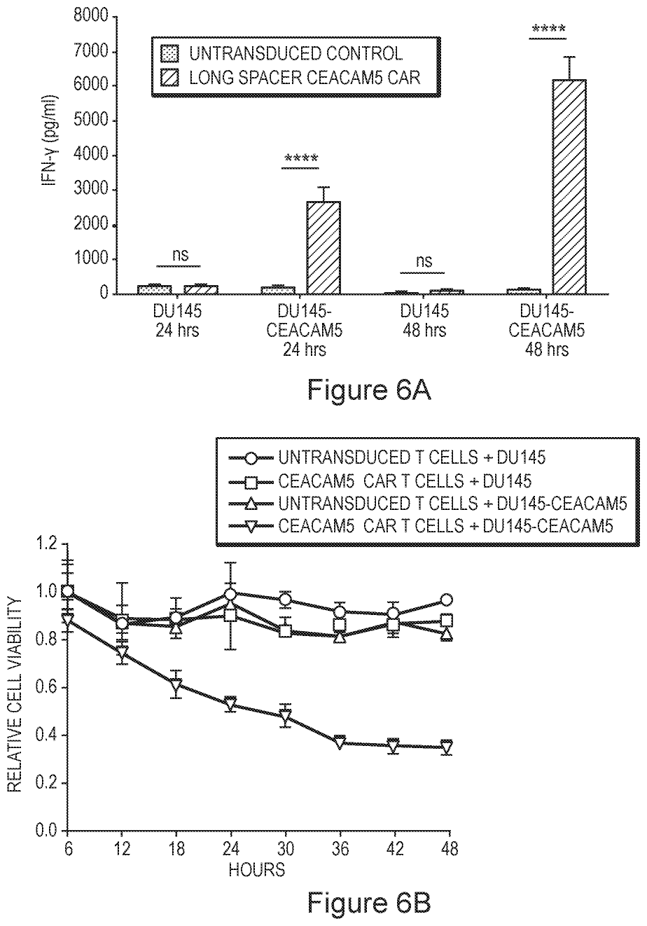

To quantify cytotoxicity, co-culture assays in an Incucyte ZOOM (See Artymovich K & Appledorn, Methods Mol Biol., 2015, 1219:35-42, incorporated by reference in its entirety), a live cell imaging and analysis system allowing for direct enumeration of effector and target cells based on bright-field and fluorescence imaging were performed. Varying effector-to-target ratios of T cells transduced with the long spacer CEACAM5 CAR and either MSKCC EF1 (CEACAM5-negative) or NCI-H660 (CEACAM5-positive) target NEPC cell lines engineered to express green fluorescent protein (GFP) were co-cultured. Target cell counts were calculated and plotted to show relative target cell viability over time in co-culture with effector cells. Co-culture of long spacer CEACAM5 CAR-transduced T cells with NCI-H660 led to >80-90% cell kill by 48 hours at effector-to-target ratios of 1:1 and 2:1 (FIG. 5B). In contrast, co-culture with the MSKCC EF1 caused a minor reduction in target cell viability by 48 hours, due to low levels of CEACAM5 expression in the MSKCC EF1 NEPC cell line (FIG. 5A). Similar co-culture studies were also performed with the prostate adenocarcinoma cell line DU145 (CEACAM5-negative) and DU145-CEACAM5 (engineered to express CEACAM5). Long spacer CEACAM5 CAR-transduced T cells had negligible effects on the DU145 cells but induced significant T cell activation and target cell death when co-cultured with DU145-CEACAM5 cells (FIGS. 6A and 6B).

These data indicate that a CEACAM5 CAR-based targeting strategy is effective in reducing viability of NEPC cells.

### Example 3: Therapeutic Targeting of CEACAM5 in Small Cell Cancers

A number of cancer cell lines (e.g., small cell lung cancer (SCLC), small cell carcinoma of the pancreas (SCCP), small cell prostate cancer) are screened for the surface expression of CEACAM5 using flow cytometry. For cancer cell lines that are CEACAM5 positive, a co-culture with CEACAM5-CAR-T cells is performed. Human peripheral blood mononuclear cells (PBMCs) from donors is obtained and activated with anti-CD3/anti-CD28 dynabeads. After four days, PBMCs are transduced with the CEACAM5-CAR. Following transduction and removal of dynabeads (seven days after activation), the CAR-T cells are used for co-culture with target cell lines (e.g., small cell lung cancer (SCLC), small cell carcinoma of the pancreas (SCCP), small cell prostate cancer) that express the CEACAM5 antigen. Varying effector-to-target ratios of target cells to T cells are tested, and cytotoxicity is measured by Incucyte live cell image analysis. Antigen-specific release of IFN-γ is analyzed in the supernatant by ELISA after 24 and 48 hrs in co-culture.

### Example 4: IFN-γ Release Using Additional CARS with Alternative Co-Stimulatory Domains

Additional lentiviral CEACAM5 CAR constructs encoding a single chain variable fragment (scFv) derived from labetuzumab (See Stein R & Goldenberg D M, Mol Cancer Ther., 2004, 3:1559-1564, which is incorporated by reference in its entirety; other suitable anti-CEACAM5 antibodies are described in International Pat. Pub. No. WO2014079886, which is incorporated by reference in its entirety), hinge/spacer, CD28 transmembrane domain, 4-1BB co-stimulatory domain, and CD3ζ activation domain (FIG. 7A), or CEACAM5 CAR constructs encoding a single chain variable fragment (scFv) derived from labetuzumab, hinge/spacer, CD28 transmembrane domain, CD28 co-stimulatory domain, 4-1BB co-stimulatory domain, and CD3ζ activation domain (FIG. 7B) were generated. The corresponding CDR sequences of labetuzumab are presented in SEQ ID NOs:1-6. The exemplary CEACAM5 CARs described in FIGS. 7A and 7B differed based on the presence of either a short spacer (IgG4 hinge) or a long spacer (IgG4 hinge and CH2+CH3 spacer). T cells expanded from human peripheral blood mononuclear cells were transduced with the various CAR constructs (Long spacer-CS1=Anti-CEACAM5-long spacer-CD28-CD3ζ); short spacer-CS2=Anti-CEACAM5-short spacer-4-1BB-CD3ζ); long spacer-CS2=Anti-CEACAM5-long spacer-4-1BB-CD3ζ); short spacer-CS3=Anti-CEACAM5-short spacer-CD28-4-1BB-CD3ζ); long spacer-CS3=Anti-CEACAM5-long spacer-CD28-4-1BB-CD3ζ) and co-culture assays with target human prostate adenocarcinoma cell line DU145 (CEACAM5-negative) and DU145-CAECAM5 (engineered to express CEACAM5 and green fluorescent protein (GFP)) were performed at a fixed effector-to-target ratio of 1:1.

Analysis of the supernatant at 24 and 48 hours by interferon-gamma (IFN-γ) ELISA revealed that antigen-specific IFN-γ release associated with the long spacer-CS2 and long spacer-CS3 CARs that had alternative co-stimulatory domains was comparable to the long spacer-CS1 CARs with CD28 as co-stimulatory domain (FIG. 8). As discussed in Example 2, this experiment also demonstrated that the short spacer CEACAM5 CARs did not increase the antigen-specific IFN-γ release, indicating that a longer spacer is useful for optimal target binding and T cell activation under the experimental conditions tested.

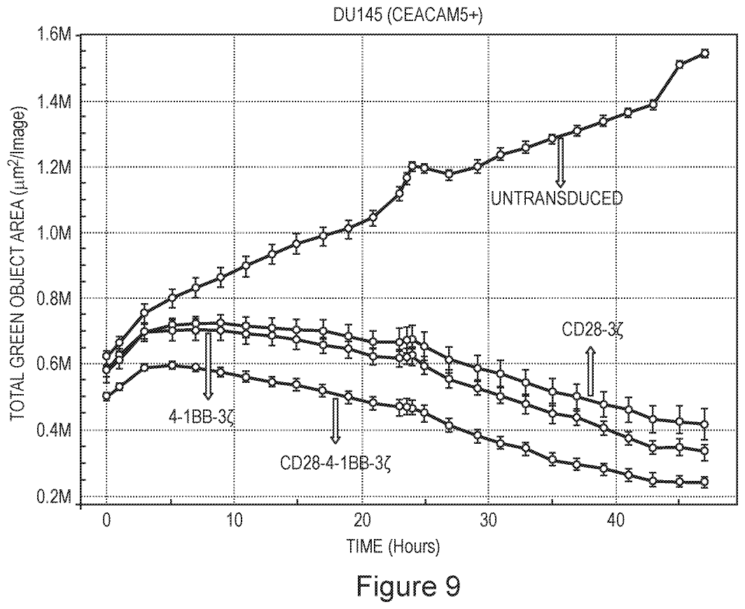

As discussed in Example 2, cytotoxicity was quantified in the co-culture assays in an Incucyte ZOOM, a live cell imaging and analysis system allowing for direct enumeration of effector and target cells based on bright-field and fluorescence imaging were performed. Varying effector-to-target ratios of T cells transduced with various long spacer CEACAM5 CARs and either DU145 (CEACAM5-negative) or DU145-CEACAM5 (CEACAM5-positive) target prostate adenocarcinoma cell lines engineered to express green fluorescent protein (GFP) were co-cultured. FIG. 9 shows the cytotoxicity results from the time course co-culture experiment.

These data indicate that CEACAM5 CARs with CD28, 4-1BB, or CD28-4-1BB as co-stimulatory domains function in a similar manner

SEQUENCE LISTING

| SEQ ID NO: | AMINO ACID SEQUENCE |
|---|---|
| SEQ ID NO: 1 | GFDFTTY |
| SEQ ID NO: 2 | HPDSST |
| SEQ ID NO: 3 | LYFGFPWFAY |
| SEQ ID NO: 4 | KASQDVGTSVA |
| SEQ ID NO: 5 | WTSTRHT |
| SEQ ID NO: 6 | QQYSLYRS |
| SEQ ID NO: 7 | EVQLVESGGGVVQPGRSLRLSCSASGFDFTTYWMSWVRQAPGKG |
| | LEWIGEIHPDSSTINYAPSLKDRFTISRDNAKNTLFLQMDSLRPEDT |
| | GVYFCASLYFGFPWFAYWGQGTPVTVSSGGGGSGGGGSGGGGSD |
| | IQLTQSPSSLSASVGDRVTITCKASQDVGTSVAWYQQKPGKAPKL |
| | LIYWTSTRHTGVPSRFSGSGSGTDFTFTISSLQPEDIATYYCQQYSL |
| | YRSFGQGTKVEIKRESKYGPPCPPCPAPEFLGGPSVFLFPPKPKDTL |
| | MISRTPEVTCVVVDVSQEDPEVQFNWYVDGVEVHNAKTKPREEQ |
| | FNSTYRVVSVLTVLHQDWLNGKEYKCKVSNKGLPSSIEKTISKAK |
| | GQPREPQVYTLPPSQEEMTKNQVSLTCLVKGFYPSDIAVEWESNG |
| | QPENNYKTTPPVLDSDGSFFLYSRLTVDKSRWQEGNVFSCSVMHE |
| | ALHNHYTQKSLSLSLGKMFWVLVVVGGVLACYSLLVTVAFIIFW |
| | VRSKRSRGGHSDYMNMTPRRPGPTRKHYQPYAPPRDFAAYRSGG |
| | GRVKFSRSADAPAYQQGQNQLYNELNLGRREEYDVLDKRRGRDP |
| | EMGGKPRRKNPQEGLYNELQKDKMAEAYSEIGMKGERRRGKGH |
| | DGLYQGLSTATKDTYDALHMQALPPR |

1. A method of treating a subject having neuroendocrine prostate cancer (NEPC), comprising administering to the subject an infusion of immune cells comprising a chimeric antigen receptor (CAR) comprising a CEACAM5 antigen-binding moiety, a transmembrane domain, and an immune cell activation moiety, wherein the immune cell activation moiety comprises one or more signaling domains.

2. The method of claim 1, wherein the neuroendocrine prostate cancer is CEACAM5⁺ neuroendocrine prostate cancer, the immune cells are CD8⁺ T cells, and the immune cells comprise a CAR comprising a CEACAM5 scFv antigen-binding moiety, a spacer domain having a length of 200 to 300 amino acids, a transmembrane domain, and an immune cell activation moiety comprising one or more signaling domains.

3. The method of claim 1, wherein the neuroendocrine prostate cancer is CEACAM5⁺ neuroendocrine prostate cancer.

4. The method of claim 1, wherein the infusion of immune cells comprises T cells.

5. The method of claim 4, wherein the T cells are CD3⁺ T cells.

6. The method of claim 5, wherein the T cells are CD8⁺ T cells.

7. The method of claim 1, wherein the immune cells are natural killer (NK) cells.

8. The method of claim 1, wherein the immune cells are natural killer T (NKT) cells.

9. The method of claim 1, wherein the CEACAM5 antigen binding moiety comprises an antibody or antigen-binding fragment thereof.

10. The method of claim 9, wherein the antibody or antigen-binding fragment thereof comprises the CDRs of labetuzumab.

11. The method of claim 10, wherein the antibody or antigen-binding fragment thereof comprises: a VH-CDR1 comprising the sequence set forth in SEQ ID NO:1; a VH-CDR2 comprising the sequence set forth in SEQ ID NO:2; a VH-CDR3 comprising the sequence set forth in SEQ ID NO:3; a VL-CDR1 comprising the sequence set forth in SEQ ID NO:4; a VL-CDR2 comprising the sequence set forth in SEQ ID NO:5; and a VL-CDR3 comprising the sequence set forth in SEQ ID NO:6.

12. The method of any of claims 9 to 11, wherein the antigen-binding fragment is a Fab or an scFv.

13. The method of claim 12, wherein the antigen-binding fragment is an scFv.

14. The method of claim 13, wherein the antigen-binding fragment is an scFv derived from labetuzumab.

15. The method of any of the preceding claims, wherein the transmembrane domain is a CD28 transmembrane domain or a CD8a transmembrane domain.

16. The method of claim 15, wherein the transmembrane domain is a CD28 transmembrane domain.

17. The method of any of the preceding claims, wherein the one or more signaling domains is selected from the group consisting of a co-stimulatory domain and an immunoreceptor tyrosine-based activation motif (ITAM)-containing signaling domain.

18. The method of claim 17, wherein the immune cell activation moiety comprises one or more co-stimulatory domains.

19. The method of claim 18, wherein the co-stimulatory domain comprises a CD28 co-stimulatory domain, a 4-1BB co-stimulatory domain, an OX40 co-stimulatory domain, or an ICOS co-stimulatory domain.

20. The method of claim 19, wherein the co-stimulatory domain comprises a CD28 co-stimulatory domain.

21. The method of any of claims 17 to 20, wherein the immune cell activation moiety comprises an ITAM-containing signaling domain.

22. The method of claim 21, wherein the ITAM-containing signaling domain comprises a CD3ζ signaling domain or an FcRγ signaling domain.

23. The method of claim 22, wherein the ITAM-containing signaling domain comprises a CD3ζ signaling domain.

24. The method of claim 17, wherein the immune cell activation moiety comprises a 28-ΔIL2RB-z(YXXQ) domain.

25. The method of claim 1, wherein the CAR further comprises a spacer domain.

26. The method of claim 25, wherein the spacer domain has a length of 1 to 500 amino acids.

27. The method of claim 26, wherein the spacer domain has a length of 200 to 300 amino acids.

28. The method of claim 27, wherein the spacer domain has a length of 229 amino acids.

29. The method of any of claims 25 to 28, wherein the spacer domain comprises a hinge domain from an immunoglobulin.

30. The method of claim 29, wherein the hinge domain from an immunoglobulin comprises the hinge domain from IgG1, IgG2, IgG3, or IgG4.

31. The method of claim 30, wherein the hinge domain from an immunoglobulin comprises the hinge domain from human IgG4.

32. The method of any of claims 25 to 31, wherein the spacer domain comprises the CH2-CH3 domain from an immunoglobulin.

33. The method of claim 32, wherein the spacer domain comprises a hinge domain from an immunoglobulin and the CH2-CH3 domain from an immunoglobulin.

34. The method of any of claims 25 to 31, wherein the spacer domain comprises the extracellular domain of CD8a.

35. The method of claim 34, wherein the spacer domain comprises a hinge domain from an immunoglobulin and the extracellular domain of CD8a.

36. The method of any of the preceding claims, wherein the CAR comprises an scFv derived from labetuzumab, a hinge of human IgG4, a CH2-CH3 domain of an immunoglobulin, a CD28 transmembrane domain, a CD28 co-stimulatory domain, and a CD3ζ signaling domain, optionally wherein the CH2-CH3 domain is a human IgG4 CH2-CH3 domain.

37. The method of any of the preceding claims, wherein the CAR comprises the amino acid sequence set forth in SEQ ID NO:7.

38. The method of any of the preceding claims, wherein the CAR increases interferon gamma (IFNγ) release by the immune cells.

39. The method of any of the preceding claims, wherein the immune cells are autologous immune cells.

40. The method of any one of claims 1 to 38, wherein the immune cells are allogeneic immune cells.

41. The method of any of the preceding claims, wherein the immune cells are administered to the subject intravenously.

42. A method of reducing or eliminating NEPC cancer cells in a subject having NEPC, comprising contacting the NEPC cancer cells with an infusion of immune cells comprising a chimeric antigen receptor (CAR) comprising a CEACAM5 antigen-binding moiety, a transmembrane domain, and an immune cell activation moiety, wherein the immune cell activation moiety comprises one or more signaling domains.

43. The method of claim 42, wherein the NEPC cancer cells comprise CEACAM5⁺ NEPC cancer cells.

44. The method of claim 42 or 43, wherein the immune cells are T cells.

45. The method of claim 44, wherein the T cells are CD3⁺ T cells.

46. The method of claim 44, wherein the T cells are CD8⁺ T cells.

47. The method of claim 42 or 43, wherein the immune cells are natural killer (NK) cells.

48. The method of claim 42 or 43, wherein the immune cells are natural killer T (NKT) cells.

49. The method of any of claims 42 to 48, wherein the CEACAM5 antigen binding moiety comprises an antibody or antigen-binding fragment thereof.

50. The method of claim 49, wherein the antibody or antigen-binding fragment thereof comprises the CDRs of labetuzumab.

51. The method of claim 50, wherein the antibody or antigen-binding fragment thereof comprises: a VH-CDR1 comprising the sequence set forth in SEQ ID NO:1; a VH-CDR2 comprising the sequence set forth in SEQ ID NO:2; a VH-CDR3 comprising the sequence set forth in SEQ ID NO:3; a VL-CDR1 comprising the sequence set forth in SEQ ID NO:4; a VL-CDR2 comprising the sequence set forth in SEQ ID NO:5; and a VL-CDR3 comprising the sequence set forth in SEQ ID NO:6.

52. The method of any of claims 49 to 51, wherein the antigen-binding fragment is a Fab or an scFv.

53. The method of claim 52, wherein the antigen-binding fragment is an scFv.

54. The method of claim 53, wherein the antigen-binding fragment is an scFv derived from labetuzumab.

55. The method of any of claims 42 to 54, wherein the transmembrane domain is a CD28 transmembrane domain or a CD8a transmembrane domain.

56. The method of claim 55, wherein the transmembrane domain is a CD28 transmembrane domain.

57. The method of claims 42 to 56, wherein the one or more signaling domains is selected from the group consisting of a co-stimulatory domain and an immunoreceptor tyrosine-based activation motif (ITAM)-containing signaling domain.

58. The method of claim 57, wherein the immune cell activation moiety comprises one or more co-stimulatory domains.

59. The method of claim 58, wherein the co-stimulatory domain comprises a CD28 co-stimulatory domain, a 4-1BB co-stimulatory domain, an OX40 co-stimulatory domain, or an ICOS co-stimulatory domain.

60. The method of claim 59, wherein the co-stimulatory domain comprises a CD28 co-stimulatory domain.

61. The method of any of claims 57 to 60, wherein the immune cell activation moiety comprises an ITAM-containing signaling domain.

62. The method of claim 61, wherein the ITAM-containing signaling domain comprises a CD3ζ signaling domain or an FcRγ signaling domain.

63. The method of claim 62, wherein the ITAM-containing signaling domain comprises a CD3ζ signaling domain.

64. The method of claim 57, wherein the immune cell activation moiety comprises a 28-ΔIL2RB-z(YXXQ) domain.

65. The method of any of claims 42 to 64, wherein the CAR further comprises a spacer domain.

66. The method of claim 65, wherein the spacer domain has a length of 1 to 500 amino acids.

67. The method of claim 66, wherein the spacer domain has a length of 200 to 300 amino acids.

68. The method of claim 67, wherein the spacer domain has a length of 229 amino acids.

69. The method of any of claims 65 to 68, wherein the spacer domain comprises a hinge domain from an immunoglobulin.

70. The method of claim 69, the hinge domain from an immunoglobulin comprises the hinge domain from IgG1, IgG2, IgG3, or IgG4.

71. The method of claim 70, wherein the hinge domain from an immunoglobulin comprises the hinge domain from human IgG4.

72. The method of any of claims 65 to 71, wherein the spacer domain comprises the CH2-CH3 domain from an immunoglobulin.

73. The method of claim 72, wherein the spacer domain comprises a hinge domain from an immunoglobulin and the CH2-CH3 domain from an immunoglobulin.

74. The method of any of claims 65 to 71, wherein the spacer domain comprises the extracellular domain of CD8a.

75. The method of claim 74, wherein the spacer domain comprises a hinge domain from an immunoglobulin and the extracellular domain of CD8a.

76. The method of any of claims 42 to 75, wherein the CAR comprises an scFv derived from labetuzumab, a hinge of human IgG4, a CH2-CH3 domain of an immunoglobulin, a CD28 transmembrane domain, a CD28 co-stimulatory domain, and a CD3ζ signaling domain, optionally wherein the CH2-CH3 domain is a human IgG4 CH2-CH3 domain.

77. The method of any of claims 42 to 76, wherein the CAR comprises the amino acid sequence set forth in SEQ ID NO:7.

78. The method of any of claims 42 to 77, wherein the CAR increases interferon gamma (IFNγ) release by the immune cells.

79. The method of any of claims 42 to 78, wherein the immune cells are autologous immune cells.

80. The method of any of claims 42 to 78, wherein the immune cells are allogeneic immune cells.

81. A method of treating a subject having small cell cancer, comprising administering an infusion of immune cells comprising a chimeric antigen receptor (CAR) comprising a CEACAM5 antigen-binding moiety, a transmembrane domain, and an immune cell activation moiety, wherein the immune cell activation moiety comprises one or more signaling domains.

82. The method of claim 81, wherein the small cell cancer is at least one of lung, prostate, pancreas, and stomach small cell cancer.

83. The method of claim 81 or 82, wherein the small cell cancer is CEACAM5 positive.

84. The method of any of claims 81 to 83, wherein the infusion of immune cells comprises T cells.

85. The method of claim 84, wherein the T cells are CD3⁺ T cells.

86. The method of claim 84, wherein the T cells are CD8⁺ T cells.

87. The method of any of claims 81 to 83, wherein the immune cells are natural killer (NK) cells.

88. The method of any of claims 81 to 83, wherein the immune cells are natural killer T (NKT) cells.

89. The method of any of claims 81 to 88, wherein the CEACAM5 antigen binding moiety comprises an antibody or antigen-binding fragment thereof.

90. The method of claim 89, wherein the antibody or antigen-binding fragment thereof comprises the CDRs of labetuzumab.

91. The method of claim 90, wherein the antibody or antigen-binding fragment thereof comprises: a VH-CDR1 comprising the sequence set forth in SEQ ID NO:1; a VH-CDR2 comprising the sequence set forth in SEQ ID NO:2; a VH-CDR3 comprising the sequence set forth in SEQ ID NO:3; a VL-CDR1 comprising the sequence set forth in SEQ ID NO:4; a VL-CDR2 comprising the sequence set forth in SEQ ID NO:5; and a VL-CDR3 comprising the sequence set forth in SEQ ID NO:6.

92. The method of any of claims 89 to 91, wherein the antigen-binding fragment is a Fab or an scFv.

93. The method of claim 92, wherein the antigen-binding fragment is an scFv.

94. The method of claim 93, wherein the antigen-binding fragment is an scFv derived from labetuzumab.

95. The method of any of claims 81 to 94, wherein the transmembrane domain is a CD28 transmembrane domain or a CD8a transmembrane domain.

96. The method of claim 95, wherein the transmembrane domain is a CD28 transmembrane domain.

97. The method of any of claims 81 to 96, wherein the one or more signaling domains is selected from the group consisting of a co-stimulatory domain and an immunoreceptor tyrosine-based activation motif (ITAM)-containing signaling domain.

98. The method of claim 97, wherein the immune cell activation moiety comprises one or more co-stimulatory domains.

99. The method of claim 98, wherein the co-stimulatory domain comprises a CD28 co-stimulatory domain, a 4-1BB co-stimulatory domain, an OX40 co-stimulatory domain, or an ICOS co-stimulatory domain.

100. The method of claim 99, wherein the co-stimulatory domain comprises a CD28 co-stimulatory domain.

101. The method of any of claims 97 to 100, wherein the immune cell activation moiety comprises an ITAM-containing signaling domain.

102. The method of claim 101, wherein the ITAM-containing signaling domain comprises a CD3ζ signaling domain or an FcRγ signaling domain.

103. The method of claim 102, wherein the ITAM-containing signaling domain comprises a CD3ζ signaling domain.

104. The method of claim 97, wherein the immune cell activation moiety comprises a 28-ΔIL2RB-z(YXXQ) domain.

105. The method of any of claims 81 to 104, wherein the CAR further comprises a spacer domain.

106. The method of claim 105, wherein the spacer domain has a length of 1 to 500 amino acids.

107. The method of claim 106, wherein the spacer domain has a length of 200 to 300 amino acids.

108. The method of claim 107, wherein the spacer domain has a length of 229 amino acids.

109. The method of any of claims 105 to 108, wherein the spacer domain comprises a hinge domain from an immunoglobulin.

110. The method of claim 109, wherein the hinge domain from an immunoglobulin comprises the hinge domain from IgG1, IgG2, IgG3, or IgG4.

111. The method of claim 110, wherein the hinge domain from an immunoglobulin comprises the hinge domain from human IgG4.

112. The method of any of claims 105 to 111, wherein the spacer domain comprises the CH2-CH3 domain from an immunoglobulin.

113. The method of claim 112, wherein the spacer domain comprises a hinge domain from an immunoglobulin and the CH2-CH3 domain from an immunoglobulin.

114. The method of any of claims 105 to 111, wherein the spacer domain comprises the extracellular domain of CD8a.

115. The method of claim 114, wherein the spacer domain comprises a hinge domain from an immunoglobulin and the extracellular domain of CD8a.

116. The method of any of claims 81 to 115, wherein the CAR comprises an scFv derived from labetuzumab, a hinge of human IgG4, a CH2-CH3 domain of an immunoglobulin, a CD28 transmembrane domain, a CD28 co-stimulatory domain, and a CD3ζ signaling domain, optionally wherein the CH2-CH3 domain is a human IgG4 CH2-CH3 domain.

117. The method of any of claims 81 to 116, wherein the CAR comprises the amino acid sequence set forth in SEQ ID NO:7.

118. The method of any of claims 81 to 117, wherein the CAR increases interferon gamma (IFNγ) release by the immune cells.

119. The method of any of claims 81 to 118, wherein the immune cells are autologous immune cells.

120. The method of any of claims 81 to 118, wherein the immune cells are allogeneic immune cells.

121. The method of any of claims 81 to 120, wherein the immune cells are administered intravenously.
